(19) United States
(12) Patent Application Publication
(10) Pub. No.: US 2021/0022892 A1
(43) Pub. Date: Jan. 28, 2021

(54) METHOD OF MANUFACTURING PLASTIC STENT USING PLASMA

(71) Applicant: BCM Co., Ltd., Gyeonggi-do (KR)

(72) Inventor: Byung Cheol MYUNG, Gyeonggi-do (KR)

(21) Appl. No.: 16/938,647

(22) Filed: Jul. 24, 2020

(30) Foreign Application Priority Data
Jul. 26, 2019 (KR) 10-2019-0091267

(51) Int. Cl. A61F 2/82; B05D 3/00; B05D 3/14; B08B 7/02

(52) U.S. Cl. CPC A61F 2/82; B05D 3/002; A61F 2002/041; B08B 7/028; B05D 3/144

(57) ABSTRACT

A method of manufacturing a plastic stent according to an embodiment of the present invention includes a first process of cleaning a surface of the stent including a plastic material to perform pretreatment, a second process of plasma-treating the pretreated surface of the stent, and a third process of introducing a hydrophilic functional group to the plasma-pretreated surface of the stent.

### CROSS REFERENCE TO RELATED APPLICATION

The present application claims priority based on Korean Patent Application No. 10-2019-0091267, filed on Jul. 26, 2019, the entire content of which is incorporated herein for all purposes by this reference.

### BACKGROUND OF THE INVENTION

### 1. Field of the Invention

The present invention relates to a plastic stent. More particularly, the present invention relates to a plastic stent for reducing the formation of a bacterial biofilm and a biliary sludge and lumen stenosis using a surface modification technology.

### 2. Description of the Related Art

A stent is a cylindrical piece of medical material used to normalize the flow of blood or body fluids when inserted into a narrowed or blocked area under X-ray fluoroscopy without surgical operation when blood or body fluids do not flow smoothly in the blood vessels, gastrointestinal tract, or 25 bile duct due to the occurrence of malignant or benign diseases.

The term “stent” has been applied worldwide in the field of interventional radiology. In recent years, however, this term has been understood primarily to mean a tubular structure to create or maintain an open state of the lumen.

Malignant bile duct obstruction may be caused by a variety of malignant diseases, such as pancreatic cancer, papillary cancer, cholangiocarcinoma, gallbladder cancer, and lymph node metastasis to the periphery of the malignant bile ducts or metastatic carcinoma therearound. Stenting has been widely performed as a conventional treatment method over a malignant-stenosed portion for the purpose of improving the quality of life by alleviating jaundice and improving systemic health during the remaining survival period in patients with malignant bile duct obstructions, which are not surgically treatable.

Typically, there are two types of stents that have been used for the purpose of biliary drainage with respect to pancreatic and bile duct diseases: one is a plastic stent and the other is a metal stent.

The plastic stent is easier to manipulate and remove and is economical compared to the metal stent, but has drawbacks in that the lumen diameter is small and the patency period is short. Further, it is known that the plastic stent is easily closed by biliary sludge and is closely related to the formation of a bacterial biofilm. Blockage of the plastic stent is related to biliary sludge and bacterial organisms associated with mixed bacterial infections and dietary fiber.

Several attempts have been made to overcome the drawbacks of plastic stents and maintain adequate biliary drainage for long periods of time. However, attempts to increase the long-term drainage effectiveness by changing the shape or material of the plastic stent have not shown a great effect. In a study comparing the diameters of plastic stents, the effect of drainage was not enhanced at diameters above 10 Fr. In addition, several attempts have been made to focus on improving bioresistance to biliary sludge and bacterial biofilms in an effort to overcome blockage of plastic stents.

Among in-vitro studies conducted using plastic stents coated with hydrophilic polymer materials, focusing on the effects of attached bacteria and biofilm formation on the patency of plastic stents, there is disclosed a technology in which a plastic stent coated with a hydrophilic polymer material reduced the attachment of bacteria and biofilm formation compared to a control group. However, even in this case, several studies conducted in vivo did not confirm prolonging of the patency period of the plastic stent. This is due to the damage caused by the wire used during endoscopic retrograde cholangio-pancreatography, in which a plastic stent is inserted, or because the coating including hydrophilic polymer materials is very fragile in practice, resulting in decomposition over time in vivo.

Therefore, there is a need for a technique for reducing the attachment of bacteria and biofilm formation when using plastic stents.

The matter described as the background technology is only for improving understanding of the background of the present invention, and it should not be accepted as acknowledging that it corresponds to the prior art already known to those skilled in the art.

### PRIOR ART DOCUMENT

### Patent Document

(Patent Document 1) Patent Document 001: Korean Patent No. 10-1430339 (2014 Aug. 13)

### SUMMARY OF THE INVENTION

Accordingly, the present invention has been made keeping in mind the above problems occurring in the related art, and an object of the present invention is to provide a method of manufacturing a plastic stent so as to reduce the formation of a bacterial biofilm and biliary sludge on the surface thereof and lumen stenosis.

In order to accomplish the above object, an embodiment of the present invention provides a method of manufacturing a plastic stent. The method includes a first process of cleaning the surface of the stent including a plastic material to perform pretreatment, a second process of plasma-treating the pretreated surface of the stent, and a third process of introducing a hydrophilic functional group to the plasma-pretreated surface of the stent.

The first process may include depositing the stent including the plastic material in a 70˜80% ethyl alcohol solution and radiating ultrasonic waves thereto.

The second process may be performed using plasma of 550 to 600 V for 5 to 7 minutes while moisture and oxygen gas are supplied to a chamber.

The third process may include depositing the plastic stent in a reaction solution for introducing a functional group and then performing plasma treatment, followed by additional deposition in the reaction solution and then drying.

The plasma treatment in the third process may be performed using a plasma of 550 to 600 V for 5 to 7 minutes while moisture and oxygen gas are supplied to a chamber.

Meanwhile, in a plastic stent according to an embodiment of the present invention, the surface thereof is modified using plasma treatment so as to impart hydrophilicity thereto.

The surface may be modified using the plasma treatment so that a hydrophilic functional group is attached to the surface.

The plasma treatment may include a first process of cleaning the surface of the stent including a plastic material to perform pretreatment, a second process of plasma-treating the pretreated surface of the stent, and a third process of introducing the hydrophilic functional group to the plasma-pretreated surface of the stent.

The method of manufacturing a plastic stent according to the present invention has the following effects.

The hydrophilicity of the surface of a plastic stent is improved, thus preventing biological contamination. Therefore, it is possible to reduce the formation of a bacterial biofilm and biliary sludge on the surface and to reduce the incidence of lumen stenosis compared to a plastic stent having an untreated surface. The reduction in the formation of the bacterial biofilm and biliary sludge and the reduction in lumen stenosis result in a reduction in damage to surrounding tissues while the plastic stent is embedded, making it safer to use in bile ducts than ordinary plastic stents.

Further, there is a merit in that the replacement period is increased due to the increased patency period.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a view schematically showing an animal experimentation process for confirming the effect of the present invention;

FIG. 2 is a view showing the results of blood tests on animals 1 month after a PE plastic stent is inserted;

FIG. 3 is a view showing the results of blood tests on animals 3 months after a PE plastic stent is inserted;

FIG. 4 is a view showing the results of blood tests on animals 3 months after a PE plastic stent is inserted;

FIG. 5 shows the results of a comparison of patency rates and biofilm and sludge rates obtained by comparing transversal cross-sections and longitudinal cross-sections of a hydrophilic PE plastic stent having a modified surface and a PE plastic stent having a non-modified surface in experimental animals monitored for 1 month after the insertion of PE plastic stents;

FIG. 6 shows the results of a comparison of patency rates and biofilm and sludge rates obtained by comparing transversal cross-sections and longitudinal cross-sections of a hydrophilic PE plastic stent having a modified surface and a PE plastic stent having a non-modified surface in experimental animals monitored for 3 months after the insertion of PE plastic stents;

FIG. 7 shows the results of a comparison of patency rates and biofilm and sludge rates obtained by comparing transversal cross-sections and longitudinal cross-sections of a hydrophilic PE plastic stent having a modified surface and a PE plastic stent having a non-modified surface in experimental animals monitored for 5 months after the insertion of PE plastic stents;

FIG. 8 is a photograph showing the cross-sections of a hydrophilic PE plastic stent having a modified surface and a PE plastic stent having a non-modified surface in experimental animals monitored for 1 month;

FIG. 9 is a photograph showing the cross-sections of a hydrophilic PE plastic stent having a modified surface and a PE plastic stent having a non-modified surface in experimental animals monitored for 3 months;

FIG. 10 is a photograph showing the cross-sections of a hydrophilic PE plastic stent having a modified surface and a PE plastic stent having a non-modified surface in experimental animals monitored for 5 months; and

FIG. 11 is a scanning electron microscopic view showing the cross-sections of PE plastic stents embedded during different periods.

### DESCRIPTION OF THE PREFERRED EMBODIMENTS

The terminology used herein is used for reference only to describe specific embodiments, and is not intended to limit the present invention. The singular forms used herein include plural forms unless the phrases clearly indicate otherwise. As used herein, the meaning of “comprising” embodies certain properties, regions, integers, steps, actions, elements and/or components, and does not exclude the existence or addition of other specific properties, regions, integers, steps, actions, elements, components and/or groups.

Although not defined otherwise, all terms including technical terms and scientific terms used herein have the same meanings as those generally understood by those skilled in the art to which the present invention pertains. Additionally, commonly used dictionary-defined terms are to be interpreted as having meanings consistent with related technical documents and the presently disclosed content, and are not to be interpreted according to ideal or very formal meanings unless so defined.

The present invention is mainly characterized by plasma treatment to impart hydrophilicity to the surface of a plastic stent. Plasma surface treatment using plasma is an environmentally non-polluting and energy-saving process, and may cause a physical-chemical characterization reaction only on the surface of a polymer while protecting the basic physical properties thereof, thereby providing various effects.

A method of manufacturing a plastic stent according to the present invention includes a first process of cleaning the surface of the stent including a plastic material to perform pretreatment, a second process of plasma-treating the pretreated surface of the stent, and a third process of introducing a hydrophilic functional group to the plasma-pretreated surface of the stent.

A variety of polymer materials may be used in the stent including the plastic material, but polyethylene is typically used as the material.

The first process is a process for cleaning the surface of the stent including the plastic material. This is to remove impurities that may be attached to the surface. The cleaning may be performed in various ways. Specifically, the stent may be deposited in an ethyl alcohol solution used as a cleaning solution, and ultrasonic waves may then be applied thereto. The concentration of the ethyl alcohol may be about 70 to 80%. Through the use of ultrasonic waves, it is possible to prevent unintended reactions that may be caused by foreign substances when plasma treatment is performed later.

The second process is a treatment process for adding a plasma to the surface of the cleaned plastic stent to thus introduce a functional group. As the plasma apparatus that is used, a direct-discharge electrode device and a low-vacuum-plasma apparatus using 40 to 60 kHz AC power are used. First, oxygen gas is injected along with moisture into a chamber at about 20 sccm (standard cubic centimeters per minute) while being exhausted, and the pressure in the chamber is maintained at about 100 mTorr. After that, the stent is treated with a plasma of 550 to 600 V for 5 to 7 minutes, taken out, deposited in an ethyl alcohol solution, and left at room temperature for 2 hours, followed by completely drying the same in a dryer.

The third process is a process for providing a functional group capable of imparting hydrophilicity to the surface of the plastic stent. The stent is deposited in the reaction solution for providing the functional group to the material.

The reaction solution is capable of being applied without limitation, as long as the reaction solution is capable of introducing a hydrophilic reaction group to the surface of the plastic stent. For example, it is possible to introduce a hydrophilic polymer to the surface thereof. A hydrophilic monomer reaction solution may be used for the purpose of manufacturing hydrophilic polymers. As the reaction solution, a solution containing an acryl-based polymer may be used. In this case, the surface may be coated with the hydrophilic polymer.

Examples of polymer resins that may be used include polymer resins having a hydrophilic functional group, such as an amino group, a carboxyl group, a hydroxyl group, a sulfonic acid group, a phosphoric acid group, and a carbonyl group.

Specific examples thereof include gums in a water-soluble polymer form, methylcellulose, alginate, starch, gelatin, casein, polyvinyl methyl ether, polyvinyl alcohol, polyvinyl acetate resins, polyacrylic acid, polyethylene glycol, polypyrrolidone, hydroxy ethylcellulose polyvinyl acetate co-crotonic acid, polyvinyl phosphonic acid, polyvinyl sulfate potassium salt, polyvinyl sulfonate sodium salt, polyvinyl alcohol boronic acid, polyvinyl alcohol ethylene ethylene, polyanethol sulfonic acid sodium salt, which is a sulfonic acid-based polymer, polysodium4styrene sulfonic acid, poly4styrene sulfonic acid sodium comaleate salt, glucomannan, xanthan gum, sodium alginate, guar gum, carboxymethyl ether sodium salt, ethyl ether, ethyl hydroxyethyl ether, hydroxyethyl ether, methylhydroxyethyl ether, dextrin, carboxymethylcellulose, poly2ethyl2oxazoline, poly2isopropenyl2oxazoline comethyl methacrylate, 2dodecenyl succinpolyglycerol, glycerol propoxylate, acrylic acid polymer, maleic acid polymer, polyacrylamide, polyacrylic acid soda, polysulfonic acid, and polyacrylic acid.

Resins, such as polysulfonic acid and polyacrylic acid, which have a hydrophilic functional group, such as OH, COOH, SO₄H, CO, and C—O—C, bonded to a carbon chain thereof may be used as a hydrophilic polymer resin.

Such a hydrophilic polymer is any one hydrophilic acryl-based polymer selected from the group consisting of polyacrylonitrile, polyacrylic acid, and polyacrylate, or any one selected from the group consisting of derivatives, in which C₁ to C₁₀ alkyl groups or C₁ to C₁₀ alkoxy groups are substituted in the polymer, and copolymers and blends thereof.

It is possible to use other hydrophilic polymers in the reaction solution. Polymer solutions having a hydrophilic functional group, such as PVA (polyvinyl alcohol), PEO (polyethylene oxide), PVP (polyvinyl pyrrolidone), and PEGMEA (polyethylene glycol methyl ether acetate) may be used.

The reaction solution may contain various catalysts. A platinum compound catalyst or a silicon compound catalyst may be used.

The plastic stent is deposited in the reaction solution, plasma-treated twice, further deposited in the reaction solution, and sonicated for 1 to 5 minutes. Finally, after the sonication is finished, the plastic stent is deposited in alcohol, left for about 4 hours, dried, allowed to react at about 60 [symbol] for about 1 hour, and cooled. During the reaction time, a hydrophilic reaction group may be introduced to the plastic surface.

Hereinafter, the present invention will be described in more detail with reference to Examples. The Examples are intended to illustrate the present invention in more detail, and the scope of the present invention is not limited to the Examples.

### Example 1

Manufacture of Plastic Stent

A central-bend-type plastic stent having a thickness of 10 Fr and a length of 90 mm was manufactured using a commercially available polyethylene (PE) material. The manufactured prototype plastic stent was subjected to a surface modification process using a vacuum plasma, thus manufacturing a hydrophilic plastic stent.

In order to form a reactive surface in a composite process using reactive treatment and plasma treatment, a polyethylene (PE) material plastic was subjected to an ultrasonic pretreatment cleaning process using 70-80% ethyl alcohol. Next, plasma pretreatment was performed with a direct discharge electrode device and a low-vacuum plasma apparatus using 40-60 kHz AC power. Oxygen gas was injected along with moisture into a chamber at 20 sccm (standard cubic centimeters per minute) while being exhausted, and the pressure in the chamber was maintained at 100 mTorr. After that, the plastic was treated with a plasma of 550 to 600 V for 5 to 7 minutes, taken out, deposited in alcohol, and left at room temperature for 2 hours, followed by completely drying the same in a dryer. After deposition in a reaction solution to which platinum (Pt) and silicon (Si) catalyst compounds and other catalysts were added, additional plasma treatment was repeatedly performed twice in the same manner as above, followed by sonication for 1 to 5 minutes in a state of deposition in the reaction solution. Finally, after the sonication is finished, the resultant plastic was deposited in alcohol, left for 4 hours, dried, allowed to react at 60° C. for 1 hour, and cooled. As described above, the polyethylene plastic stent treated using the plasma is modified at a surface thereof so as to have a hydrophilic property due to the presence of a hydrophilic functional group.

### Experimental Example 1

Measurement of Contact Angle

In order to confirm that the surface of the polyethylene plastic stent treated using a plasma was modified, water droplets were dropped onto the polyethylene plastic stent to measure a contact angle using a Krüss Drop Shape Analyzer (DSA 10, Krüss GmbH, Hamburg, Germany). The contact angle was measured using a sessile drop technique. The contact angle was smaller in the case of the polyethylene plastic stent subjected to a plasma treatment process for hydrophilic surface modification than in the case of a control polyethylene plastic stent not treated with the plasma. Further, the surface roughness of the lumen was reduced in the case of the polyethylene plastic stent treated with the plasma compared to the case of the control polyethylene plastic stent.

### Experimental Example 2

Animal Experiment

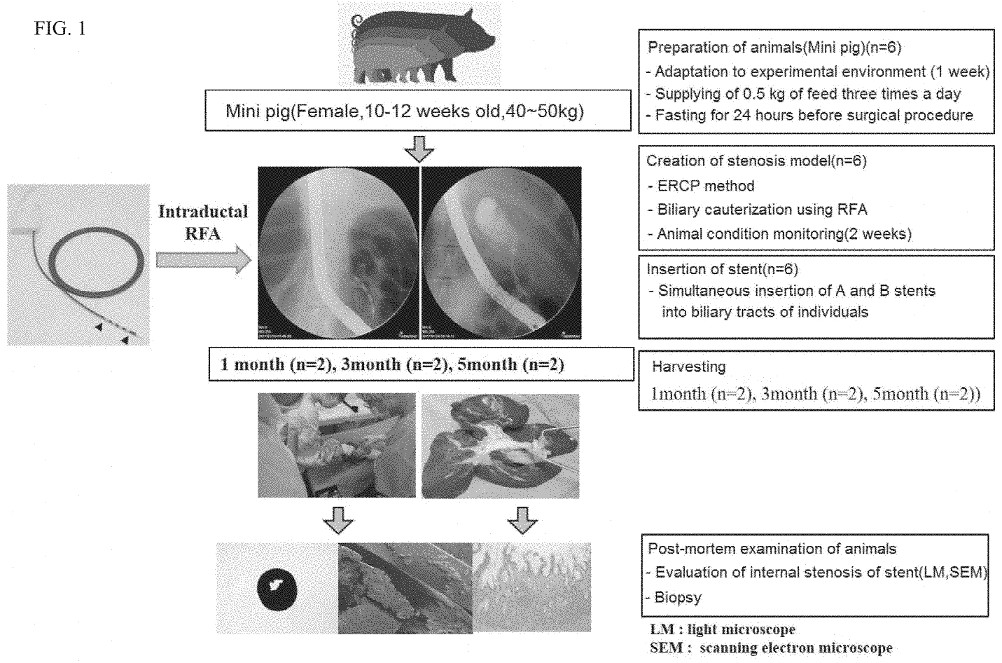

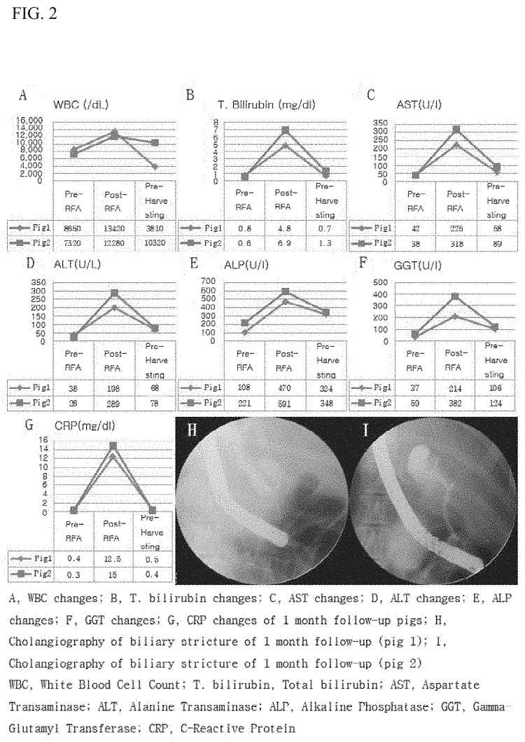

FIG. 1 is a view schematically showing an animal experimentation process for confirming the effect of the present invention. The animal experiment was broadly divided into four steps (FIG. 2). A first step is a step of preparing experimental animals, which is a step of allowing the experimental animals to adapt to the test environment before the experiment after the experimental animals are obtained. A second step is a stenosis model formation step, which is a step of monitoring the state of the experimental animals for two weeks after biliary cauterization by an intraductal radio-frequency ablation electrode (RFA) using endoscopic retrograde cholangio-pancreatography. A third step is a step of inserting a plastic stent after confirmation of animal stenosis using a C-arm fluoroscope. In the fourth and final step of harvesting the experimental animals, two experimental animals were harvested at each of 1 month, 3 months, and 5 months.

1) Preparation of Experimental Animals

A total of six animals of 10- to 12-week-old female micro pig M-type (micro pig M-type; Medi Kinetics Co., Ltd, Pyeongtaek, Gyeonggi-do, Korea) having a mean weight of 50 kg were used as subjects. Before the start of the experiment, a one-week adaptation period was ensured, and only healthy animals were used for animal experiments. In all of the experiments, the animals were bred in an animal breeding room in which a temperature of 23±2° C., a relative humidity of 50±5%, a ventilation number of 10 to 12 times/hour, a lighting time of 08:00 to 20:00, and an intensity of illumination of approximately 400 lux were set. During the acclimation period and the experimental period, one animal was put into one cage, solid feed (Purina) was supplied once before the start of business and once at 4:00 pm, that is, twice a day. The solid feed was supplied in an amount of 0.8 to 1.2 kg for one supplying. After fasting for 24 hours the day before the surgical procedure, the experiment was performed. This study was reviewed and approved by the Animal Experimental Ethics Committee of the Samsung Life Sciences Research Institute, which is a Certification Authority for AAALAC International (Association for Assessment and Accreditation of Laboratory Animal Care International), and was conducted in accordance with the guidelines for the management and use of experimental animals set forth by the committee (IACUC Approval Number: 20160712001).

2) Creation of Experimental Animal Model for Biliary Stenosis

A total of six female micro pig M-type animals were randomly assigned into groups each including two animals so as to be monitored for 1 month, 3 months, and 5 months. A biliary stenosis model using biliary cauterization of an intraductal radio-frequency ablation electrode was performed according to the method presented by Shin J U et al. of the present research team. The experiment was performed the next day after fasting for 24 hours before the surgical procedure of the biliary stenosis model. On the day of the surgical procedure, the experimental animals were injected intramuscularly with 50 mg/ml Ketamine® and 20 mg/kg and zolazepam (Zoletil®; 6 mg/kg) and sedated using xylazine (Rompun®; 2 mg/kg) by a veterinary surgeon, and tracheal intubation was then performed. After the tracheal intubation, anesthesia was maintained using 2% isoflurane. The electrocardiogram, heart rate, blood pressure, oxygen saturation, and end-tidal carbon dioxide (CO₂) partial pressure thereof were monitored by a veterinary surgeon. Enrofloxacin (2.5 mg/kg) was injected intramuscularly until two days before the surgical procedure in order to prevent cholangitis caused by the surgical procedure. On the day of the surgical procedure, ketoprofen (2 mg/kg) was administered intramuscularly for the purpose of pain control. After a TJF240 (Olympus America, Inc, Melville, N.Y.), which is a therapeutic endoscope, was inserted, a duodenal papilla was checked. Under a fluoroscope, the surgical procedure was performed according to a wire-guided cannulation method for performing selective cannulation of a biliary catheter using a wire. After that, the papilla was expanded along the wire using a hurricane balloon catheter (Boston Scientific Corp., 10 mm diameter), and an intraductal radio-frequency ablation electrode was then inserted into a common bile duct. Cauterization was performed at 10 W and 80 C for 90 sec using the intraductal radio-frequency ablation electrode (ELRA electrode; STARmed Co. Ltd, Goyang, Gyeonggi-do, Korea) embedded in the common bile duct.

3) Confirmation of Biliary Stenosis in Experimental Animals and Insertion of Stent

Two weeks after experimental animals were subjected to intraductal radio-frequency ablation (RFA), biliary stenosis was confirmed using a biliary fluoroscope using 25 ml of a contrast agent after duodenal papilla cannulation using a TJF240 endoscope. In a blood test, the use of WBC (white blood cells), AST (aspartate transaminase), ALT (alanine transaminase), ALP (alkaline phosphatase), GGT (gamma-glutamyl transferase), and CRP (C-reactive protein) was included. The blood test was performed three times, i.e., before and after the surgical procedure of the stenosis model, and at the final follow-up. Under the biliary fluoroscope, two PE plastic stents were embedded into the biliary tract using a 0.035-inch wire (hydrophilic tipped guidewire, Boston Scientific Corp., Natick, USA). The PE plastic stents were embedded so that the proximal tips of the PE plastic stents were located in different branches of the intrahepatic bile ducts.

4) Harvesting of Experimental Animals

1 month, 3 months, and 5 months after the polyethylene plastic stents were inserted, the two pigs in each group were injected intramuscularly with 50 mg/ml Ketamine® and 20 mg/kg zolazepam (Zoletil®; 6 mg/kg) and were sedated using xylazine (Rompun®; 2 mg/kg) by a veterinary surgeon, and tracheal intubation was then performed, as on the day of the surgical procedure. After the tracheal intubation, anesthesia was maintained using 2% isoflurane. The electrocardiogram, heart rate, blood pressure, oxygen saturation, and end-tidal carbon dioxide (CO₂) partial pressure thereof were monitored by a veterinary surgeon. Open laparotomy of all pigs was performed by one very skilled veterinary surgeon. Median incision was performed and the duodenum was excised. The excised duodenum was dissected in a longitudinal direction to harvest the PE plastic stent. The internal stenosis of the harvested PE plastic stent, along with the patency rate and the biofilm and biliary sludge thereon, were measured. A biopsy was performed to compare histological scores.

In all six experimental animal pigs (micro pigs), a biliary stenosis model using an intraductal radio-frequency ablation electrode was successfully created. Further, a total of 12 plastic stents (vacuum-plasma-process-surface-modified hydrophilic plastic stent, N=6; normal plastic stent, N=6) were successfully inserted without complications related to surgical procedures, such as bleeding or perforation, into all six experimental animals that had successfully undergone the surgical procedure of biliary stenosis. All experimental animals survived during the harvesting time without complications after the insertion of the PE plastic stent and after the surgical procedure. After the two experimental animals in each group were sacrificed at times of 1 month, 3 months, and 5 months, the patency rate and biofilm and sludge rate of the PE plastic stent were evaluated.

Evaluation of Patency Rate and Biofilm of Plastic Stent

The extent of lumen stenosis of the harvested polyethylene plastic stent after experimenting in the biliary of two pigs in each group at times of 1 month, 3 months, and 5 months was analyzed using an optical microscope and a scanning electron microscope. To this end, a patency rate (%) and a biofilm and sludge rate (%) were used in the present experiment. The patency rate is defined as the ratio of the luminal area (Luminal Area_Test) of the harvested PE plastic stent occupied in the luminal area (Luminal Area_Base) of the polyethylene plastic stent measured before the experiment. Therefore, the value obtained by dividing the luminal area (Luminal Area_Test) of the harvested PE plastic stent by the luminal area (Luminal Area_Base) of the PE plastic stent measured before the experiment is multiplied by 100 to obtain a patency rate value in units of %. The patency rate of the PE plastic was calculated using the following equation from the optical microscopic images of the longitudinal and transversal cross-sections of the PE plastic stent using ImageJ 1.47v.

$$Patency rate . \%$$

$$Patency Rate ( \% ) = \frac{( Luminal {Area}_{\_ Test} )}{Luminal {Area}_{\_ Base}} \times 100$$

The biofilm and sludge rate (%) is defined as the ratio of the biofilm and biliary sludge occupied in the luminal area (Luminal Area_Base) of the PE plastic stent measured before the experiment. It is difficult to accurately distinguish and measure the biofilm and biliary sludge using an optical microscope when the luminal area of the PE plastic stent is measured. Accordingly, the biofilm and sludge rate was obtained to perform quantitative comparison. Therefore, the biofilm and sludge rate is defined as the ratio of the biofilm and sludge area (Luminal Area_Test) of the harvested PE plastic stent occupied in the luminal area (Luminal Area_Base) of the PE plastic stent measured before the experiment. Therefore, the value obtained by dividing the biofilm and sludge area (Luminal Area_Test) of the harvested PE plastic stent by the luminal area (Luminal Area_Base) of the PE plastic stent measured before the experiment is multiplied by 100 to obtain the biofilm and sludge rate value in units of %. Likewise, the biofilm and sludge rate was calculated using the following equation from the optical microscopic images of the longitudinal and transversal cross-sections of the plastic stent using ImageJ 1.47v.

$$Biofilm and sludge rate . \%$$

$$Biofilm and sludge Rate ( \% ) = \frac{( Luminal {Area}_{\_ Test} )}{Luminal {Area}_{\_ Base}} \times 100$$

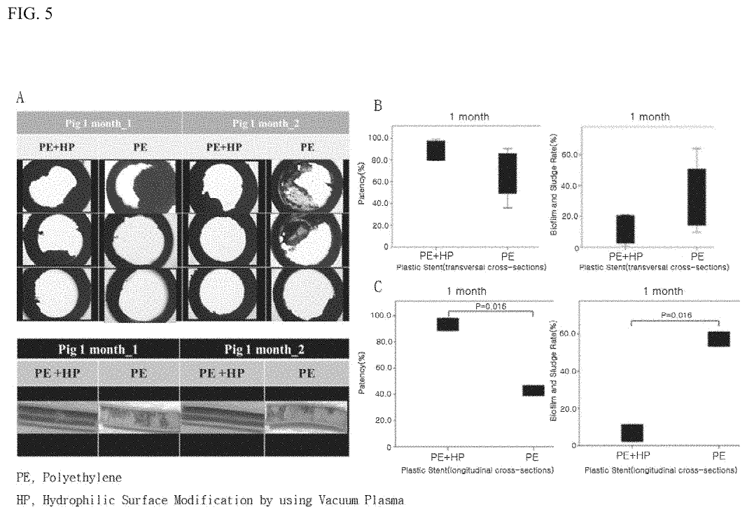

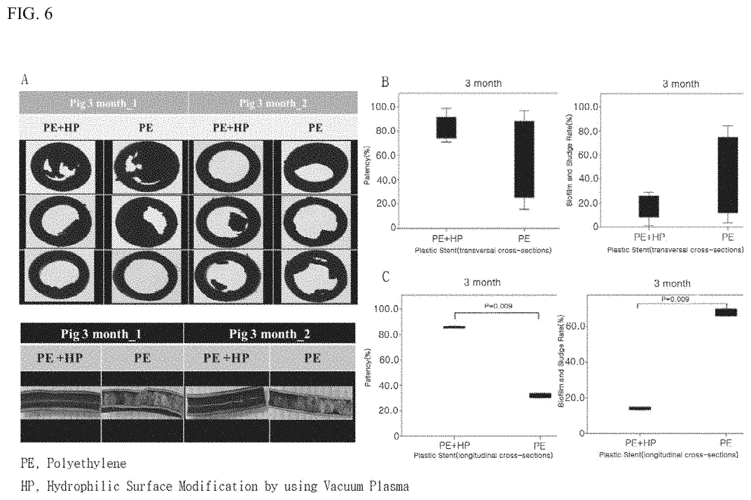

The PE plastic stent harvested from the pig's biliary was fixed to a specially manufactured frame and was then cut at intervals of 10 mm using R35 ether disposable microtome blades (Feather Safety Razor Co., Osaka, Japan) (FIGS. 5A and B). After the tips of both ends of the PE plastic stent, which were cut at intervals of 10 mm, were cut at intervals of 1 mm, the inside of the tube of the plastic stent was observed using an optical microscope (FIG. 5C). Through observation using the optical microscope, the luminal patency rate and biofilm of the PE plastic stent were quantitatively measured. To this end, ImageJ 1.47v (National Institute of Health, Bethesda, Md., USA) was used (FIG. 6). In order to measure the base area (Luminal Area_Base), the area of the hydrophilic PE plastic stent, which was manufactured so as to be modified at a surface thereof using a vacuum plasma process, and the area of a control group were measured before the surgical procedure.

In order to perform observation using the optical microscope, the segments that remained after the PE plastic stent was cut were cut at intervals of 4 mm. After the surface of the cut PE plastic stent was coated with platinum (Pt), the inside of the PE plastic stent was observed using a scanning electron microscope (SEM, S-4800; Hitachi, Tokyo, Japan). The extent of luminal patency and biofilm and biliary sludge of the PE plastic stent were observed using a scanning electron microscope, thereby accomplishing qualitative observation.

All experiments were performed three times or more in the same manner for measurement. A hierarchical linear model was used to compare the patency rates and the biofilm and sludge rates of the PE plastic drainage tube treated using a surface modification process and a control PE plastic drainage tube. As a result of the analysis, in the case where the p value was less than 0.05 (p-value <0.05), the case was defined as a statistically significant result. IBM SPSS version 24.0 (IBM Corp., Armonk, N.Y., USA) was used as a statistical program.

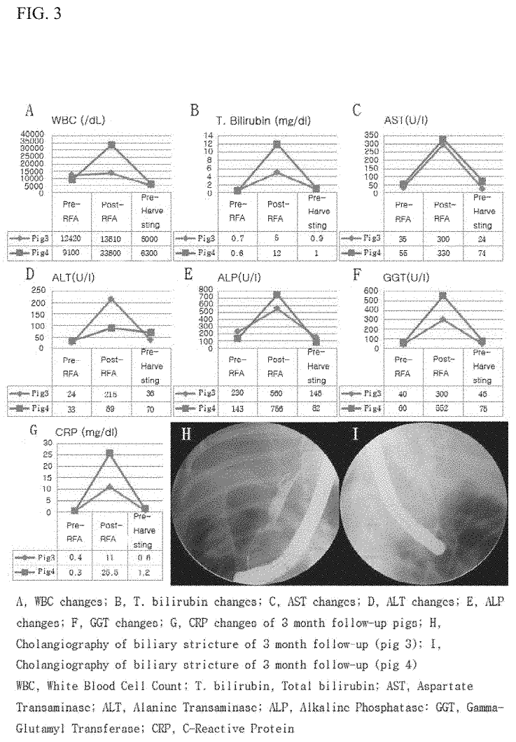

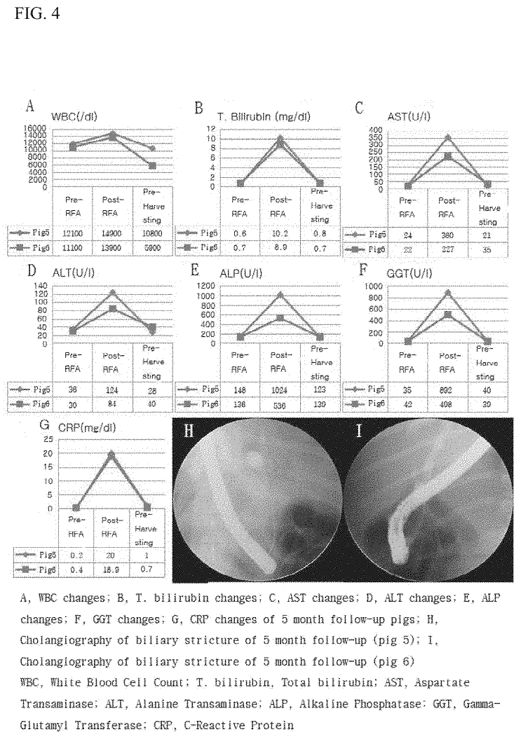

The results of blood tests on the experimental animals 1 month, 3 months, and 5 months after the PE plastic stent was inserted were divided into a pre-stenosis procedure, a post-stenosis procedure (when the PE plastic stent was inserted), and pre-harvesting of experimental animals, which are shown in the drawings. FIG. 2 is a view showing the results of blood tests on animals 1 month after the PE plastic stent is inserted. FIG. 3 is a view showing the results of blood tests on animals 3 months after the PE plastic stent is inserted. FIG. 4 is a view showing the results of blood tests on animals 3 months after the PE plastic stent is inserted.

Referring to FIGS. 2 to 4, in all of the cases of 1 month, 3 months, and 5 months, it was observed that WBC, AST, ALT, ALP, GGT, and CRP were elevated after the stenosis procedure but were reduced after a PE plastic tube was inserted. H and I of FIG. 2 show biliary fluoroscopic findings 2 weeks after the stenosis procedure in experimental animals 1 and 2, which were monitored for 1 month after the PE plastic stent was inserted. H and I of FIG. 3 show biliary fluoroscopic findings 2 weeks after the stenosis procedure in experimental animals 3 and 4, which were monitored for 3 months after the PE plastic stent was inserted. H and I of FIG. 4 show biliary fluoroscopic findings 2 weeks after the stenosis procedure in experimental animals 3 and 4, which were monitored for 5 months after the PE plastic stent was inserted. Successful biliary stenosis was confirmed in all six animals.

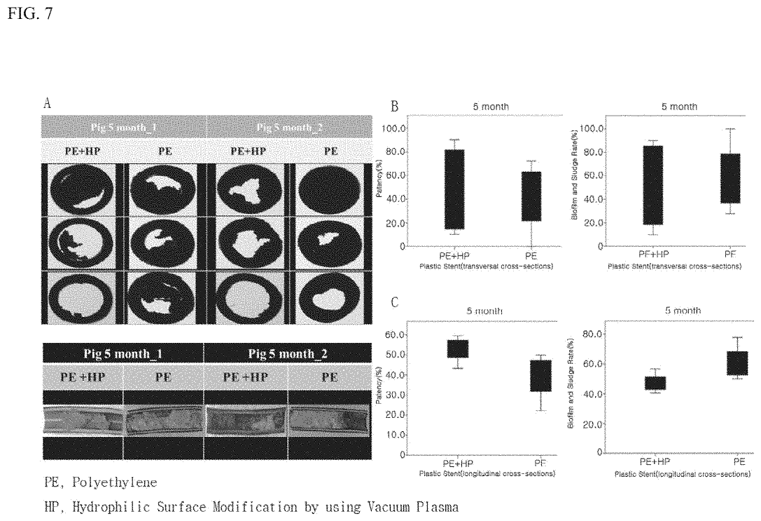

FIGS. 5 to 7 show the results of comparing patency rates and biofilm and sludge rates, obtained by comparing transversal cross-sections and longitudinal cross-sections of a hydrophilic PE plastic stent having a modified surface and a PE plastic stent having a non-modified surface in experimental animals monitored for 1 month, 3 months, and 5 months after the insertion of PE plastic stents. The transversal cross-sections and the longitudinal cross-sections of the PE plastic stents in the experimental animals monitored for 1 month were compared. In the case of the transversal cross-section, the patency rate of the hydrophilic PE plastic stent having a modified surface was high (82.4±21.3 vs. 68.3±22.9%, p=0.256). Reduced formation of the biofilm and biliary sludge was observed in the hydrophilic PE plastic stent having a modified surface (17.6±21.3 vs. 31.7±22.9%, p=0.256), but the reduction was not statistically significant. As a result of comparison of the longitudinal cross-sections of the PE plastic stents, a statistically significant improvement in patency rate was observed (93.23±6.6 vs. 42.7±5.6%, p=0.016) and reduced formation of biofilm and biliary sludge was observed (6.7±6.6 vs. 57.3±5.6%, p=0.016) in the hydrophilic PE plastic stent having a modified surface (FIG. 5). In the comparison of the transversal cross-sections and the longitudinal cross-sections of the PE plastic stents in the experimental animals monitored for 3 months, as in the result for 1 month, in the case of the transversal cross-section, the patency rate of the hydrophilic PE plastic stent having a modified surface was high (79.0±22.3 vs. 56.5±32.8%, p=0.136), and reduced formation of the biofilm and biliary sludge was exhibited in the hydrophilic PE plastic stent having a modified surface (21.0±23.0 vs. 43.5±32.8%, p=0.136) but the reduction was not statistically significant. In the case of the longitudinal cross-section, in the hydrophilic PE plastic stent having a modified surface, a statistically significant improvement in patency rate was observed (85.9±1.2 vs. 32.1±2.8%, p=0.009), and reduced formation of the biofilm and biliary sludge was observed (14.1±1.2 vs. 67.9±2.8%, p=0.009) (see FIG. 6). In the case of the transversal cross-sections of the PE plastic stents in the experimental animals monitored for 5 months, the patency rate of the hydrophilic PE plastic stent having a modified surface was high (69.1±30.3 vs. 53.4±29.5%, p=0.083), and reduced formation of the biofilm and biliary sludge was exhibited (30.9±30.3 vs. 46.6±29.5%, p=0.083) but there was no statistically significant difference. In the results of the longitudinal cross-sections, the patency rate of the hydrophilic PE plastic stent having a modified surface was high (53.0±6.9 vs. 39.5±12.1%, p=0.113), and reduced formation of the biofilm and biliary sludge was exhibited (47.0±6.9 vs. 60.5±12.1%, p=0.113), but there was no statistically significant difference. However, sites exhibiting a patency rate of 0% were observed in the transversal and longitudinal cross-sections of the PE plastic stent having a non-modified surface (see FIG. 7).

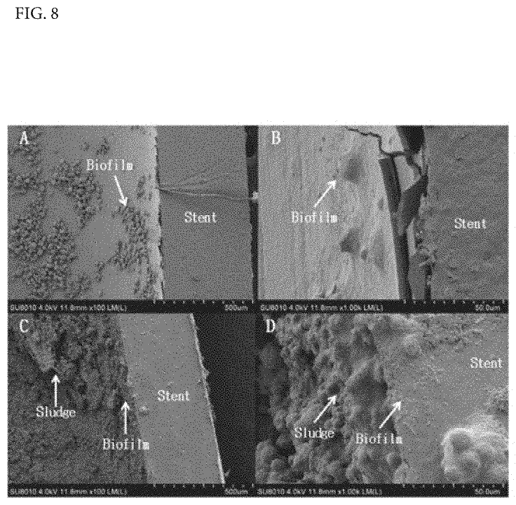

The cross-sections of the hydrophilic PE plastic stent having a modified surface and the PE plastic stent having a non-modified surface in the experimental animals monitored for 1 month, 3 months, and 5 months were observed using a scanning electron microscope. FIGS. 8 to 10 are photographs showing the cross-sections of the hydrophilic PE plastic stent having a modified surface and the PE plastic stent having a non-modified surface in the experimental animals monitored for 1 month, 3 months, and 5 months. A and B show the hydrophilic PE plastic stent having the modified surface, and C and D show the PE plastic stent that is not modified.

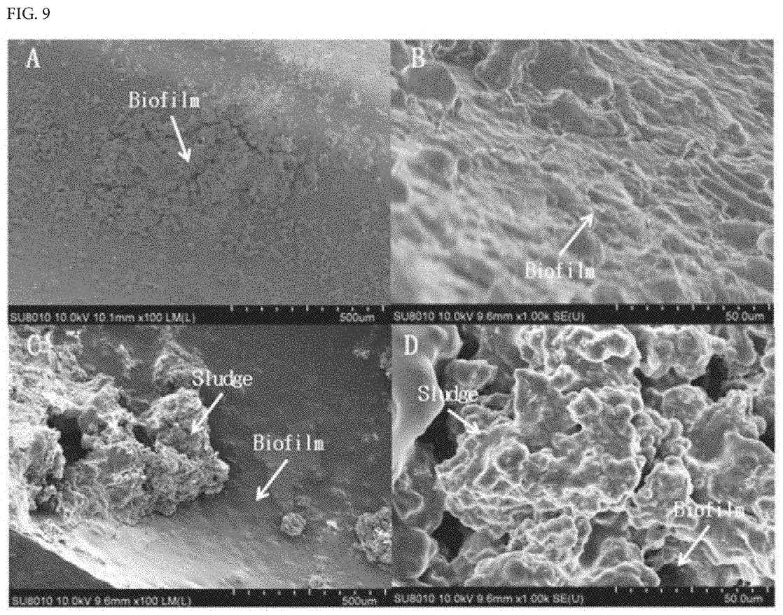

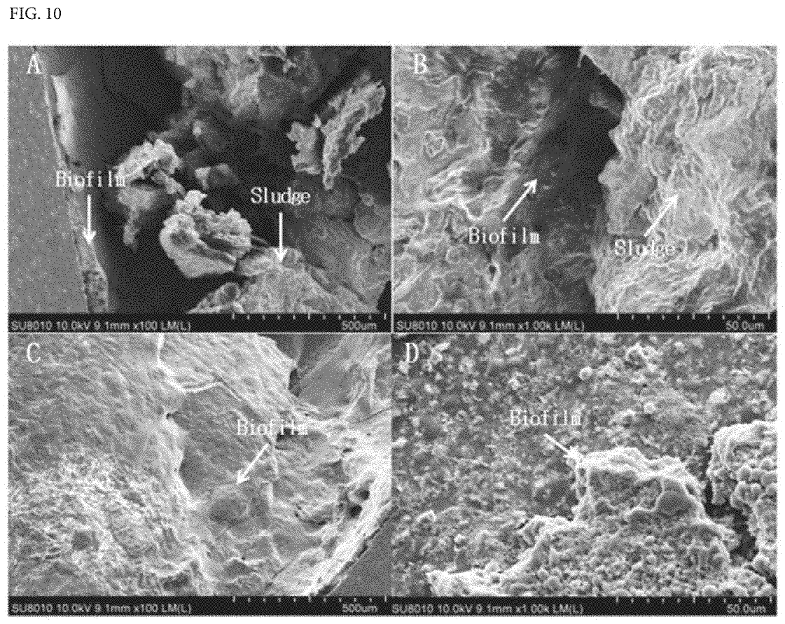

First, the cross-sections of the PE plastic stents embedded during the same period were compared using a scanning electron microscope. In the case of the experimental animals in which a PE plastic drainage tube was embedded for 1 month, it could be confirmed that significantly less biofilm and biliary sludge were formed in the case of the hydrophilic PE plastic stent having the modified surface (in FIG. 8, A (×100) and B (×1000)) than in the case of the PE plastic stent that was not modified (in FIG. 8, C (×100) and D (×1000)). As in the case of the PE plastic stent embedded for 1 month, in the case of the PE plastic stent embedded for 3 months, it was confirmed that more biofilm and biliary sludge were formed in the case of the PE plastic stent having the modified surface than in the case of the PE plastic stent having the non-modified surface (in FIG. 9, C (×100), D (×1000)). In the case of embedding for 5 months, less biofilm and biliary sludge were formed in the case of the PE plastic stent having the non-modified surface than in the case of the hydrophilic PE plastic stent having the modified surface (in FIG. 10, A (×100), B (×1000)).

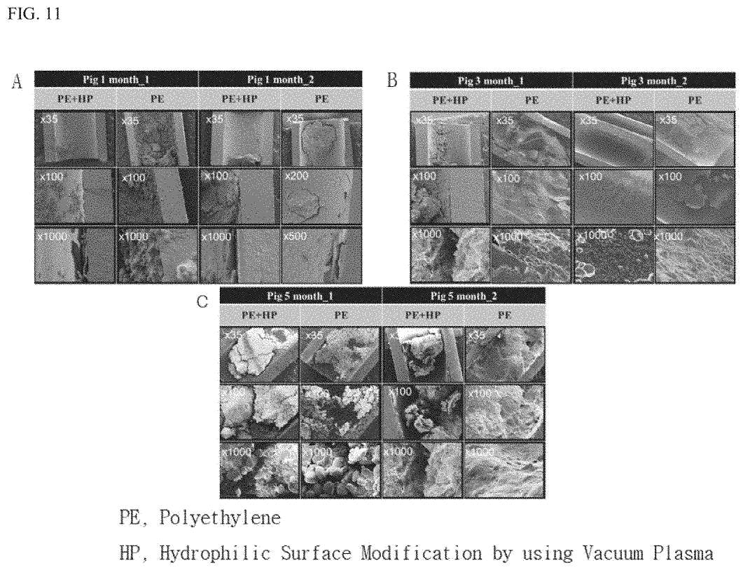

FIG. 11 is a scanning electron microscopic view showing the cross-sections of the PE plastic stents embedded during different periods. PE+HP indicates the hydrophilic PE plastic stent having the modified surface, and PE indicates the PE plastic stent that is not modified.

The biofilm and biliary sludge were formed to a greater thickness when the PE plastic stent was embedded for 3 months than when the PE plastic stent was embedded for 1 month. In the case of the PE plastic stents harvested from the experimental animals in which the PE plastic stent was embedded for 5 months, it was confirmed with the naked eye that the thickness of the biofilm and biliary sludge was remarkably larger in both the hydrophilic plastic stent having the modified surface and the plastic stent having the non-modified surface than in the cases where the plastic stent was embedded for 1 month and 3 months.

Although the embodiments of the present invention have been described with reference to the accompanying drawings, it will be understood by those skilled in the art that the present invention may be implemented in other specific forms without changing the technical spirit or essential features thereof.

Therefore, it should be understood that the embodiments described above are illustrative in all respects and not restrictive. The scope of the present invention is indicated by the claims set forth below rather than the detailed description, and it is to be construed that all changes or modified forms derived from the meaning and scope of the claims and their equivalent concepts are included in the scope of the present invention.

What is claimed is:

1. A method of manufacturing a plastic stent, the method comprising:
a first process of cleaning a surface of the stent including a plastic material to perform pretreatment;
a second process of plasma-treating the pretreated surface of the stent; and
a third process of introducing a hydrophilic functional group to the plasma-pretreated surface of the stent.

2. The method of claim 1, wherein the first process includes depositing the stent including the plastic material in a 70˜80% ethyl alcohol solution and radiating ultrasonic waves thereto.

3. The method of claim 1, wherein the second process is performed using a plasma of 550 to 600 V for 5 to 7 minutes while moisture and oxygen gas are supplied to a chamber.

4. The method of claim 1, wherein the third process includes depositing the plastic stent in a reaction solution for introducing the functional group and then performing plasma treatment, followed by additional deposition in the reaction solution and then drying.

5. The method of claim 4, wherein the plasma treatment in the third process is performed using a plasma of 550 to 600 V for 5 to 7 minutes while moisture and oxygen gas are supplied to a chamber.

6. A plastic stent, a surface of which is modified using plasma treatment so as to impart hydrophilicity thereto.

7. The plastic stent of claim 6, wherein the surface is modified using the plasma treatment, so that a hydrophilic functional group is attached to the surface.

8. The plastic stent of claim 7, wherein the plasma treatment includes:
a first process of cleaning the surface of the stent including a plastic material to perform pretreatment;
a second process of plasma-treating the pretreated surface of the stent; and
a third process of introducing the hydrophilic functional group to the plasma-pretreated surface of the stent.
